(19) United States
(12) Patent Application Publication
(10) Pub. No.: US 2010/0196297 A1
(43) Pub. Date: Aug. 5, 2010

(54) Urine as a source of alternative cellular energy pigments (ACE-pigments) in the Assesment and therapy of diseases

(76) Inventor: William John Martin, South Pasadena, CA (US)

Correspondence Address: William John Martin, 1634 Spruce Street, South Pasadena, CA, 91030, US

(21) Appl. No.: 12/381,003

(22) Filed: Mar. 5, 2009

Related U.S. Application Data
(63) Continuation of application No. 12/322,491, filed on Feb. 2, 2009, now abandoned.

(51) Int. Cl. A61K 35/22; A61P 43/00

(52) U.S. Cl. 424/63; 424/537

(57) ABSTRACT

Alternative cellular energy pigments (ACE-pigments) are over-produced in response to various types of viral infections and to other pathological conditions. ACE pigments accumulate at the sites of disease and also in other regions of the body, including the oral cavity. ACE pigments are considered to play a significant role in the body's healing response to both local and system diseases. They appear to exist in a relatively inactive form in which they can fluorescence under ultraviolet (UV) light illumination, especially if stained with a suitable dye, such as neutral red. The present invention describes a method of using ACE pigments collected from the urine of patients for the purpose of activating the patient's ACE pathway in a child with autism, with an extrapolation to patients with a wide variety of other infectious and non-infectious illnesses for which there is a presumed or shown insufficiency of cellular energy. Urine or urine-derived material containing the collected ACE pigments is placed onto a suitable material, such as a paper towel, stained with neutral red dye and induced to fluoresce using UV illumination. The fluorescing mixture is placed over various areas of the body and/or directly placed over a localized skin lesion and the UV illumination continued. This process will assist in the local as well as overall activation of the ACE pathway in the patient with resulting clinical benefit.

### CROSS REFERENCE TO CO-PENDING PATENT APPLICATIONS

Method of Using the Body's Alternative Cellular Energy Pigments (ACE-pigments) in the Therapy of Diseases. Submitted Feb. 20, 2009

Methods for Collecting and Using the Body's Alternative Cellular Energy Pigments (ACE-pigments) in the Therapy of Diseases. Submitted Feb. 2, 2009. Ser. No. 12/322,491

Methods for Detection of Ultraviolet Reactive Alternative Cellular Energy Pigments (ACE-Pigments). William John Martin Submitted Dec. 24, 2007.

Method of Assessing and of Activating the Alternative Cellular Energy (ACE) Pathway in the Therapy of Diseases. William John Martin Submitted Jan. 17, 2008

Enerceutical Mediated Activation of the Alternative Cellular Energy (ACE) Pathway in the Therapy of Diseases Submitted May 8, 2008

Method of Activating the Alternative Cellular Energy (ACE) Pathway Using Hydrogen. Submitted Jul. 9, 2008

### CROSS REFERENCE TO PREVIOUSLY SUBMITTED BUT NOW ABANDONED PATENT APPLICATION

Ser. No. 10/044,683. Therapy of stealth virus associated cancers and other conditions using light. William John Martin. (Abandoned)

Ser. No. 10/047,313. Therapy of stealth virus associated cancers and other conditions using medium chain triglycerides. William John Martin. (Abandoned)

Ser. No. 10/050,232. Diagnosing and monitoring the therapy of stealth virus infections based on the detection of auto-fluorescent material in hair. William John Martin. (Abandoned)

Ser. No. 10/058,480. Therapy of stealth virus associated cancers and other conditions using magnetic energy. William John Martin. (Abandoned)

Ser. No. 10/164,258 Energy supportive therapy of stealth virus associated diseases. William John Martin. (Abandoned)

Ser. No. 10/174,466 Sound therapy of stealth virus associated diseases. William John Martin. (Abandoned)

Ser. No. 10/192,936 ACE-Pigments and humic acids as energy sources. William John Martin. (Abandoned)

Submitted: 10/Methods for Collection of Alternative Cellular Energy Pigments (ACE-pigments). William John Martin. (Abandoned)

Submitted: 10/Methods for Elimination of Toxic Alternative Cellular Energy Pigments (ACE-pigments) and for Their Replacement Using Activated Humates, Including Humic and Fulvic Acids. William John Martin. (Abandoned)

### UNITED STATES PATENT (AWARDED)

U.S. Pat. No. 5,985,546 Stealth virus detection in the chronic fatigue syndrome. William John Martin

U.S. Pat. No. 5,891,468 Stealth virus detection in the chronic fatigue syndrome. William John Martin

U.S. Pat. No. 5,753,488 Isolated stealth viruses and related vaccines. William John Martin

U.S. Pat. No. 5,703,221 Stealth virus nucleic acids and related methods. William John Martin

U.S. Pat. No. 6,368,637. Method and composition for topical treatment of viral lesions. Jon Stoneburner

### PCT (PATENT COOPERATION TREATY)

WO 92/20797 Stealth virus detection in the chronic fatigue syndrome. William John Martin

WO 99/34019 Stealth virus nucleic acids and related methods. William John Martin

WO 99/60101 Stealth viruses and related vaccines. William John Martin

### REFERENCES TO PUBLISHED AND TO SUBMITTED ARTICLES ALTERNATIVE CELLULAR ENERGY PIGMENTS (ACE-PIGMENTS)

1. Martin W J. Alternative cellular energy pigments mistaken for parasitic skin infestations. Exp. Mol. Path 78: 212-214, 2005.
2. Martin W J. Alternative cellular energy pigments from bacteria of stealth virus infected individuals. Exp. Mol. Path 78: 217-217, 2005.
3. Martin W J. Progressive Medicine. Exp Mol Path 78: 218-220, 2005.
4. Martin W J, Stoneburner J. Symptomatic relief of herpetic skin lesions utilizing an energy based approach to healing. Exp. Mol. Path 78: 131-4, 2005.
5. Martin W J. Etheric Biology. Exp Mol Path 78: 221-227, 2005.
6. Martin W J. Stealth Virus Culture Pigments: A Potential Source of Cellular Energy. Exp. Mol. Pathol. 74: 210-223, 2003.
7. Martin W J. Complex intracellular inclusions in the brain of a child with a stealth virus encephalopathy. Exp. Mol. Pathol. 74: 179-209, 2003.
8. Martin W J. Photons and phonons: Theoretical aspects of biophysics and potential therapeutic applications. Proceeding of Neural Therapy Workshop on Sound and Light Therapy, Seattle, Wash., February 21-23, 2003.
9. Martin W J. Activation of the Alternative Cellular Energy (ACE) Pathway as Natural Therapy for Herpes Simplex and Herpes Zoster Virus Infections. Submitted electronically to Journal of Infectious Diseases on 3/198/2008 Article rejected for publication. Revised article in progress.
10. Martin W J. Activation of the Alternative Cellular Energy (ACE) Pathway as Natural Therapy for Patients with Autism. Submitted electronically to Journal of Autism and Disability Disorders on Jun. 24, 2008. Article rejected for publication. Revised article in progress.

### STEALTH ADAPTED VIRUSES

1. Martin W J Chronic fatigue syndrome among physicians. A potential result of occupational exposure to stealth viruses. Explore 2001; 10: 7-10.
2. Martin W J. Stealth Viruses. Explore 2001; 10: 17-19.
3. Durie G M, Collins R. Martin W J. Positive stealth virus cultures in multiple myeloma. A possible explanation for neuropsychiatric co-morbidity. Presented at the Am. Soc. Hematology annual meeting October 2000.
4. Martin W J. Chemokine receptor-related genetic sequences in an African green monkey simian cytomegalovirus-derived stealth virus. Exp Mol Pathol. 2000; 69:10-6.
5. Martin W J., Anderson D. Stealth virus epidemic in the Mohave Valley: severe vacuolating encephalopathy in a child presenting with a behavioral disorder. Exp Mol Pathol. 1999; 66:19-30.
6. Martin W J. Melanoma growth stimulatory activity (MGSA/GRO-alpha) chemokine genes incorporated into an African green monkey simian cytomegalovirus-derived stealth virus. Exp Mol Pathol. 1999; 66:15-8.
7. Martin W J. Bacteria-related sequences in a simian cytomegalovirus-derived stealth virus culture. Exp Mol Pathol. 1999; 66:8-14.
8 Martin W J. Stealth adaptation of an African green monkey simian cytomegalovirus. Exp Mol Pathol. 1999; 66:3-7.
9 Martin W J. Cellular sequences in stealth viruses. Pathobiology 1998; 66:53-8.
10 Martin W J. Detection of RNA sequences in cultures of a stealth virus isolated from the cerebrospinal fluid of a health care worker with chronic fatigue syndrome. Case report. Pathobiology. 1997; 65:57-60.
11 Martin W J., Anderson D. Stealth virus epidemic in the Mohave Valley. I. Initial report of virus isolation. Pathobiology. 1997; 65:51-6.
12 Martin W J. Simian cytomegalovirus-related stealth virus isolated from the cerebrospinal fluid of a patient with bipolar psychosis and acute encephalopathy. Pathobiology. 1996; 64:64-6.
13 Martin W J. Stealth viral encephalopathy: report of a fatal case complicated by cerebral vasculitis. Pathobiology. 1996; 64:59-63.
14 Martin W J. Genetic instability and fragmentation of a stealth viral genome. Pathobiology. 1996; 64:9-17.
15 Martin W J. Severe stealth virus encephalopathy following chronic-fatigue-syndrome-like illness: clinical and histopathological features. Pathobiology. 1996; 64:1-8.
16 Martin W J. Stealth virus isolated from an autistic child. J Autism Dev Disord. 1995; 25:223-4.
17 Gollard R P, Mayr A., Rice D A, Martin W J. Herpesvirus-related sequences in salivary gland tumors. J Exp Clin Cancer Res., 1996; 15: 14.
Martin W J., Glass R T. Acute encephalopathy induced in cats with a stealth virus isolated from a patient with chronic fatigue syndrome. Pathobiology. 1995; 63:115-8.
Martin W J, et al. African green monkey origin of the atypical cytopathic 'stealth virus' isolated from a patient with chronic fatigue syndrome. Clin Diag Virol 1995: 4: 93-103.
Martin W J. Stealth viruses as neuropathogens. CAP Today. 1994; 8: 67-70.
Martin W J. et al. Cytomegalovirus-related sequence in an atypical cytopathic virus repeatedly isolated from a patient with chronic fatigue syndrome. Am J Pathol. 1994; 145: 440-51.

### STATEMENT REGARDING FEDERALLY SPONSORED RESEARCH OR DEVELOPMENT

No Federal funding was received in support of the research covered in this patent application.

### REFERENCE TO SEQUENCE LISTING, A TABLE, OR A COMPUTER PROGRAM LISTING COMPACT DISK APPENDIX

None provided.

### BACKGROUND OF THE INVENTION

With the exception of chlorophyll-containing plants and bacteria, it is widely believed that virtually all of the energy produced by living organisms is derived from the catabolic (metabolic breakdown) of food. The applicant has proven that an additional source of cellular energy is available to living cells. It is derived through the conversion of physical energies, including electromagnetic radiation, magnetic fields, sound, etc., to biological energy that can be used for biosynthetic reactions and other cellular functions. This energy conversion (transduction) is mediated by materials that the applicant has termed “alternative cellular energy pigments” (ACE-pigments). These pigments were identified as intracellular inclusions that can lead to extra-cellular particles in the liquid medium of cultured cells infected with cell damaging (cytopathic) stealth adapted viruses. The viruses were termed “stealth” or “stealth-adapted” because they do not evoke an inflammatory reaction within the infected individuals. They are molecularly heterogeneous, although some are unequivocally derived from herpes viruses, including African green monkey simian cytomegalovirus (SCMV). Stealth adapted viruses have been cultured from patients with a wide range of neuropsychiatric and other illnesses.

The pigmented structures that develop in cultures of stealth adapted viruses are conglomerates of smaller microscopic and sub-microscopic particles. They can also take the form of long threads and ribbon-like structures. ACE-pigments show various properties indicative of their capacity to absorb light energy (photons). For example, the particles will typically display microscopic movements within a fluid environment, which far exceed that explainable as simple Brownian motility. These movements can be shown to be at least in part in response to the illumination delivered by the microscope. The larger particles can occasionally display slow oscillatory (back and forth) movements in response to light illumination. The smaller particles and some of the fibers and threads will commonly fluoresce slightly when exposed to ultraviolet (UV) light. Much of the emitted fluorescence is in the green to yellow range, but occasionally it is strikingly red. Even green visible light can provoke red emission from some particles. The induced fluorescence of the particles can be greatly enhanced in the presence of certain dyes, including neutral red, a tricyclic non-fluorescent dye. The particles can also differentially alter the emitted fluorescence of known UV fluorescent dyes such as acridine orange. As expected, stealth virus infected cells stained with either neutral red or acridine orange contains finely dispersed material that displays multi-colored (green, yellow and red) fluorescence, which is not seen in uninfected cells. Similar multicolor UV particulate fluorescence can often be seen in the cytoplasm of neutral red stained polymorphonuclear cells obtained from both known and presumptively stealth adapted virus infected patients.

Fluorescing materials, either alone or after mixing with neutral red dye, can also be collected directly from the skin or from perspiration of various patients. Methods to enhance perspiration such as the use of heat can increase the secretion of ACE pigments, as can the taking of a hot bath. Another collection method is to have a perspiring patient sleep without clothing between clean sheets from which the particles can be retrieved the next morning. Hair samples can have attached fluorescing materials and/or display intrinsic fluorescent properties. Fluorescing materials, especially after mixing with a small amount of neutral red dye, can also be retrieved from spittle (spit) and from other body fluids, including the urine. Discrete particles can occasionally be recognized in such fluids by the evoked fluorescence and characteristic non-Brownian movements. Long fibers and larger particles can also form in such fluids over time. The presence of neutral red or acridine orange dye with added light appears to accentuate the development of such larger forms of ACE pigments, as well as seemingly newly formed crystals.

ACE pigments are generally present the vicinity of skin lesions caused by Herpes simplex virus (HSV) and Herpes zoster virus (HZV) (hereafter occasionally lumped together as herpetic skin lesions). They also occur in association with warts caused by human papillomavirus (HPV) and are presumably part of the body's response to many other chronic infectious agents, including hepatitis viruses and HIV. Accumulations of ACE pigments have also been detected in association with other chronic skin conditions including squamous cell carcinomas, seborrheic keratosis and psoriasis. One method for detecting ACE pigments in skin lesions is by rubbing the lesion with a Q-tip or simple gauze swab, staining with a dilute solution of neutral red (typically 0.1 to 1 mg/ml) and observing for UV inducible yellowish fluorescence. Absorbent paper can also be used to collect ACE pigments from skin lesions, as well as from broader areas of the skin and hair,and from bodily fluids, including spittle and urine.

ACE pigments were identified as potential therapeutic agents when it was shown that their appearance in stealth adapted viral cultures correlated with a lessening of the cytopathic effect (CPE) caused by the viruses. Thus, for example, removal of the ACE-pigments from the cultures resulted in a rapid reactivation of the CPE that could be very easily prevented by the re-addition of isolated ACE pigments to the fresh re-feeding culture medium. Rapid cellular repair is achieved even though the added ACE pigments are essentially only present in the supernatants, suggesting a biophysical effect occurring over a distance, rather than a biochemical process that would require penetration of the particles into the cells.

The healing properties of ACE pigments was further suggested by the expedited healing that occurs when neutral red is applied to active HSV and HZV skin lesions followed by UV illumination. Prior to the discovery of ACE pigments, the success of this procedure that dates back to the 1970's was incorrectly attributed to a direct killing effect of the illuminated dye on virus particles.

Knowing the biophysical properties of ACE pigments, I reasoned that if collected ACE pigments were to be activated in close vicinity of herpes virus skin lesions they would still be able to achieve a therapeutic benefit. Such a method would avoid any direct contact of the neutral red or other dye with the herpes skin lesions. This consideration is important since some researchers as well as the US Food and Drug Administration (FDA) have raised the possibility of neutral red inducing oncogenic (cancer causing) mutations in herpes viruses. Moreover, if successful in the therapy of active herpes skin lesions, the indirect approach would likely have broader therapeutic applications to other diseases, including internal illnesses caused by stealth adapted viruses.

I have further reasoned that ACE-pigments probably exist in four distinct states: i). Directly fluorescent when exposed to UV light; ii) fluorescent only if exposed to UV light after the addition of a small quantity of a fluorescence triggering dye, such as neutral red, to the ACE pigments. The dye seemingly alters ACE pigments in a manner that allows the ACE pigments to better absorb the UV energy and retransmit some of the absorbed energy as fluorescence; iii) non-responsive to either direct or dye enhanced UV illumination because of being uncharged and no longer energy chargeable or vi) non-fluorescent with or without the dye because of being fully charged.

An observation that assisted in this four stage classification was the demonstration that UV activation of ACE pigments in the second of the above stages could achieve conversion of distantly situated second stage pigments to stage one. In other words, neutral red-UV light activation of only a portion of the ACE pigments on a Q-tip can render the remaining ACE pigments on the Q-tip capable of becoming fluorescent when directly placed under UV illumination. Similarly, activation of ACE pigments in one location of the body, for example in a herpes skin lesion can result in partial and more complete systemic activation of the body's ACE pathway. Systemic activation is shown when other areas of the patient's skin and/or parts of the patient's oral cavity acquire the property of becoming fluorescent when directly illuminated with UV light. This change commonly accompanies and follows the local UV therapy of neutral red stained herpetic skin lesions. These quite striking observations imply that some form of energy is radiating from the activated stage-two ACE pigments, which were triggered to fluoresce by using neutral red along with UV illumination. This radiated energy is seemingly capable of converting adjacent or even distantly located ACE pigments from stage two to stage one, such that they are now able to fluoresce when directly illuminated with UV light. When fully activated, the ACE pathway will apparently no longer yield ACE pigments that will fluoresce either directly with UV illumination or after the addition of neutral red dye. This is the situation expected in healthy individuals in whom there is no insufficiency of cellular energy.

Prior studies in patients with HSV skin lesions at more than one location in the body have confirmed expedited healing in all lesions even though the neutral red application along with UV illumination was limited to a single lesion. As noted above, although, not necessary for the healing of the distant skin lesions, the ACE pigments in these distant areas will fluoresce if directly examined under UV illumination during the treatment of the lesion selected for neutral red application. Even beyond distant herpes skin lesions, it is usually possible to see UV evoked fluorescence occurring in other areas of the body, such as the soles of the feet and palms of the hands. Furthermore, other skin lesions, such as keratoses, will not uncommonly appear distinct from the surrounding skin because of a heightened fluorescence in response to UV illumination. Inside the mouth is another striking region of the body that can acquire quite marked UV induced fluorescence during and after treating a patient for a localized herpes skin lesion.

Based on these observations, I concluded that a communicating network exists within the body such that it was possible to trigger systemic activation of the ACE pathway by triggering a local response at an area of pathology, or even at a normal appearing area of the body at which ACE pigments have accumulated or are placed. A restatement of this concept is that even external activation of ACE pigments removed from the body could likely impart a heightened healing capacity of the ACE pathway throughout the body, if placed elsewhere on the body and appropriately activated. The heightened healing capacity would be indicated by UV inducible fluorescence at sites of distinct skin pathology and/or areas of ACE pigment accumulations. Note that healing of distant lesions is not dependent on direct UV illumination at the distant site. With the completion of successful activation of the patient's ACE pathway, it is to be expected that UV and/or dye inducible fluorescence of ACE pigments, either collected from the patient's skin, urine, etc. or directly viewed within skin areas or in the oral cavity of the individual, will no longer occur. This is the situation expected in individual in whom there is no apparent insufficiency of cellular energy.

Based on these considerations, a formal proposal (number 071) entitled “A research study examining the effectiveness of distant activation of ACE pigments associated with recurrent human herpes simplex virus-induced skin lesions” was submitted in early 2007 to the Institute of Progressive Medicine, Institutional Review Board (IRB). The proposal received unanimous approval and allowed for the studies that led to the additional observations embodied in this and co-pending patent applications. (References to published articles on Alternative Cellular Energy Pigments (ACE-pigments) and to Stealth Adapted Viruses are listed above. Also included are a series of now abandoned and pending patent applications that provide additional documentation of the earlier findings leading to this present application. Information in all of the published references and cited awarded and abandoned patents applications of William John Martin are incorporated by reference into the present patent application.

### BRIEF SUMMARY OF THE INVENTION

The invention discloses the use ACE pigments collected from a patient's urine in the activation of the patient's ACE pathway. The invention is a simple extension of prior and ongoing studies that use either formulated products or sources of the body's ACE pigments, such as from skin lesions, perspiration, spittle, etc. for the purpose of local and/or systemic activation of the patient's ACE pathway, for the therapy of illnesses.

ACE pigments that will fluoresce under UV light illumination after mixing with a small quantity of neutral red dye can be readily collected from patients with various diseases. The sources include active herpes infections as well as from the skin, hair, and body fluids of patients without active skin lesions at the time of collection. The body fluids that can contain ACE pigments include saliva (spittle), urine, and blood (serum, plasma and leukocytes). The identification of presumptive ACE pigments in urine dates back to many years ago when I first began to stain urine samples with acridine orange and observe the stained urine microscopically for moving fluorescing particles.

Collected ACE pigments that are not fully activated are tentatively identified by their UV fluorescence, either directly or more typically when stained with a suitable dye such as acridine orange or neutral red. They are generally microscopically finely particulate in appearance and display light-inducible movements when viewed under a high power microscope. They are also generally stainable with iodine/iodide solution. The presence of retrievable UV reactive ACE pigments from a patient is currently being used as a presumptive marker for potential clinical benefit of therapeutic interventions that are aimed at enhancing the patient's ACE pathway. It is not a marker for any particular illness and indeed reactive ACE pigments have been collected from athletes accustomed to strenuous, energy depleting exercises. The optimal goal for both patients and athletes has been to restore non-fluorescence reactivity to UV light of their body's ACE pathway.

The presumptive presence of reactive ACE pigments in the urine of a patient is defined for the purpose of this application as urine that strongly fluoresce when exposed to UV light after mixing of the urine with an ACE pigment fluorescence triggering dye, such as neutral red. This is a somewhat more restricted definition than is used for the detection of ACE pigments in other body areas since some degree of weak UV fluorescence is a common characteristic of many untreated urine samples. Direct urine fluorescence is also stated to be a potential, but not reliable sign of poisoning with the antifreeze agent ethylene glycol (Parsa T., et al. The usefulness of urine fluorescence for suspected antifreeze ingestion in children. Am J Emerg Med. 2005 23:787-92, 2005)

A suitable means of UV illumination of a urine sample is a compact spiral fluorescent light that emits UV-A light. These lights are termed ProLume™ and are available from Halco Lighting (Atlanta, Ga.). The detection of fluorescence can be enhanced by viewing the UV illuminated material in the absence of visible light. The dye that is commonly used to detect ACE pigments is neutral red since it does not fluoresce by itself, yet it readily promotes the fluorescence of reactive ACE pigments. It is also less environmentally toxic than some alternative dyes such as acridine orange.

A preferred method for detecting UV reactive ACE pigments in collected urine is to add approximately 5-10% by volume of approximately 0.5 mg/ml solution of neutral red and to look for UV inducible fluorescence by directly shinning the UV light onto the surface of the urine. The surface of positive fluorescing urines can also display evidence of gas formation, presumably hydrogen and/or oxygen. These gases can add to a sense of vaporization from the surface of the urine. Negative fluorescing urine simply stain red.

The present patent application relates specifically to using collected urine as the source of ACE pigments in the therapy of diseases associated with an insufficiency of cellular energy. The representative disease chosen to illustrate this principle is autism (presumptively caused by a stealth adapted virus). This example reinforces the general principle of using collected ACE pigments in the local and systemic activation of the body's ACE pathway for the purposes of treating diseases. It also underscores the value of monitoring the body's ACE energy pathway by periodically assessing the levels of neutral red triggered fluorescence in bodily fluids.

The therapeutic uses of urine as a method of activating a patient's ACE pathway is broadly consistent with the historical but not scientifically supported belief in ingesting one's own urine or rubbing one's urine into the skin. These practices are commonly referred to as urotherapy. They are neither recognized as being safe or effective.

### BRIEF DESCRIPTION OF THE DRAWINGS

None included

### DETAILED DESCRIPTION OF THE INVENTION

Urine was collected from a 14 year old child and showed no discernable fluorescence when illuminated with a 13 watt UV-A compact light bulb. An estimated 10% by volume of an approximately 0.5 mg/ml neutral red solution was gently added and it led to no immediate fluorescence of the urine. When stirred with a Q-tip to allow full mixing of the solutions, yellow fluorescence under UV illumination became strikingly apparent. The Q-tip was similarly fluorescent when directly examined under the UV light. The urine fluorescence persisted for several hours at which time the urine was discarded. This preliminary study confirmed that the child's urine presumptively contained reactive ACE pigments. Similar results have been found in other patients.

Another sample of urine was obtained from the child and a portion added to a water dampened kitchen absorbent towel that was then placed onto a sheet of Saran™ wrap that covered the lower back area of the child. The urine soaked towel was then sprayed with a dilute solution of neutral red and illuminated with a UV-A light. The paper towel showed bright yellowish fluorescence as expected from the earlier study. The treatment was continued for 45 minutes with periodic questioning of the child by her mother.

The child chosen for this exemplary study has had periodic treatments using the enerceutical™ product “Epione”™ since May of 2008. Both she and her mother are highly reliable research subjects in that they were clearly able to report on the benefits of each therapy session. Moreover, they correctly identified various additional test batches of materials that were subsequently shown to lack the enerceutical™ activity of Epione.™ The question posed to the child and to her mother was whether the urine protocol was comparable in its benefits to that of active Epione™ and earlier studies using spittle. The answer was in the affirmative with comments from the child about experiencing similar positive feelings, as with the spittle. More pointedly, the mother witnessed several positive changes. Basically, she can reliably tell that while her child experiences a calm sleepiness during an effective therapy in contrast to the indifference when an inactive solution is being used in conjunction with neutral red dye. In response to effective therapy, her daughter quickly reverts at the end of the treatment session to a state of heightened alertness. This is typically characterized by her exhibiting renewed curiosity about herself and her surroundings; using words more meaningfully and with more emotional attachment; playing more constructively with her toys; better responding to home based schooling; and appearing better coordinated in her physical activities. Of major importance to the mother, her child also experiences very few if any absence seizures for a week or so following a successful therapy. It is clear to both mother and child that the urine therapy is an alternative to spittle, although, ascetically the latter is much preferred.

Using urine to assess the ACE pathway and for potential therapy will be of particular value in very young infants. Diapers are an obvious source of urine in infants. For some adults, psychiatric illnesses can also preclude cooperatively and understandingly providing spittle. For such patients, as well as comatose patients, urine is a preferred useful source of ACE pigments to spittle. Effective enerceutical™ formulations are clearly also indicated under such circumstances.

The previously described spittle protocol had been offered as an alternative to the use of Epione™ in patients with many other types of illnesses. By extrapolation, the urine protocol is similarly, an alternative to both Epione and spittle in therapy of this wide range of illnesses. Specifically, the illnesses include various mental and neurological illnesses, virus infections (both with and without localized skin lesions), mitochondria disorders, emphysema, and both cardiovascular and cerebrovascular conditions. Although Saran™ wrap and other forms of polyethylene sheeting are transparent; the procedure does not seemingly involve the transfer of light energy between the paper and the skin, since a light impermeable layer can be used instead of the Saran™ wrap. An advantage of a transparent layer is that by simply peeling back the paper layer, one can periodically observe any underlying lesion for UV inducible fluorescence.

The present hypothesis is that there is a coupling of the energy from the neutral red stained ACE pigments or enerceutical™ formulation on the paper towel to unstained ACE pigments in the skin. Absorption of this energy converts the body's ACE pigments from stage 2 (neutral red dependent fluorescence) to stage 1 (directly fluorescent) and then onto stage 4 (fully charged). The nature of the transferred energy(s) is under active investigation with consideration being given to a vibrational or kinetic force.

It is anticipated that both urine and spittle will become directly fluorescent as a result of secretion of stage 1 ACE pigments, whose formation will be thye result of therapy induced partial activation of a patient's ACE pathway. With more complete therapy, the body's ACE pigments should enter stage 4 and be no longer directly fluorescent. Thus urine monitoring can provide an aid in assessing the progress of whole body therapy aimed at activating the ACE pathway.

Both the spittle and urine protocols are practical alternatives to collecting ACE pigments from active HSV, HZV and other accessible viral induced skin lesions. Probing a herpetic skin lesion can be painful and it can be difficult to collect material from a lesion once it is beginning to scab. While general areas of the skin and hair can provide ACE pigments in these and other types of disease conditions, the distribution of ACE pigments can be quite irregular in contrast to the collection of spittle or urine. Moreover, assessing the neutral red inducible fluorescence of both spittle and urine should provide more information than using either source alone.

As noted above, the use of urine is not intended as a replacement of spittle, Epione™ or other suitable enerceutical™ products under most situations. Rather it simply provides an alternative, especially in very young infants and some psychiatric and/or comatose patients. It is fully anticipated that urine, or urine derived materials from one person may be used to activate the ACE pathway in some other individuals and that a direct method of ACE activating energy transfer into humans and animals will likely soon be developed. It is also clearly recognized that urine can provide a useful starting material for the biochemical and functional characterization of ACE pigments.

Various modifications and adaptations of this basic approach of using ACE pigments collected from urine to achieve overall and even local activation of the body's ACE pathway will be apparent to any practitioner who has followed this line of scientific inquiry. These include the use of other dyes; other UV light sources; etc. Furthermore, the medical conditions that can potentially be treated using the method of externally activating the ACE pigments obtained from a patient, or even from another individual, are not confined to autism or even to infectious diseases. A more basic understanding of the utility of the described ACE activation method relates to the broader concept that many individuals have an insufficiency of cellular energy to fully cope with disease processes. In one context, the ACE pathway is viewed as an adjunct to the immune system in combating various types of conventional infections, including those due to herpes viruses, papillomaviruses, hepatitis viruses, influenza, etc. The ACE pathway is even more critically involved in the defense of infections caused by stealth adapted viruses that are not effectively recognized by the cellular immune system. Similarly, the ACE pathway can potentially play an increasingly important role in the evolving immune suppression caused by infections that directly target the immune system such as HIV or in extremely rapid infections that outpace the development of an effective immune response, such as Ebola.

Autism is viewed as being part of a wide spectrum of illnesses associated with stealth adapted viruses. Approximately a third of autistic children show evidence of epilepsy and ongoing clinical studies have confirmed a reduction in seizure activity upon administering ACE therapy to autistic children. ACE therapy has also been successfully employed in treating patients with psychiatric and neurological illnesses. Some of these illnesses can also be associated with persisting infections with stealth adapted viruses. Certain disease entities, including infections with stealth adapted viruses, can be associated with the production of relatively large amounts of ACE pigments. Indeed, some of the pigments from these patients will fluorescence when directly exposed to UV light, although usually not nearly as much as when mixed with neutral red or some other dyes. The most prominent of these disease entities is currently being referred to as delusional parasitosis or Morgellon's disease. The strong electrostatic and other energy converting capacity of the ACE pigments isolated from these patients have led some observers to the mistaken belief that they are living parasites. The common psychiatric component of this illness can also explains why their physicians attribute such beliefs to delusions.

Non-infectious diseases for which the level of ACE pathway activation may play a crucial role include emphysema and other respiratory diseases in which the tissues receive insufficient oxygen for normal mitochondria based metabolism. Similarly, cellular metabolism can be restricted in the brain and heart by cerebrovascular and cardiovascular diseases, respectively. Cancer is yet another disease in which a cellular energy deficiency can be posited as limiting a healing process. The option clearly exists for testing the ACE pathway in virtually all forms of diseases and augmenting it using either the body's own ACE pigments as illustrated in this patent application or formulated sources as described in previous and co-pending patent applications.

While the major focus of prior and present studies on the ACE pathway is on humans, it is clear that the methods under study are also applicable to infectious and other insufficiency of cellular energy associated disease states in animals. The ACE pathway is also considered to be operative in plants and various plant derived materials, included in the broader category of ACE-pigments related materials that I have termed Enerceuticals.™

An important characteristic of Enerceuticals is that they do not need to localize to the site of disease pathology since they can exert a “field effect” applicable over a distance. Hence it is possible to use energy stimulated ACE pigments external to the body to activate the body's ACE pathway and to achieve therapeutic benefits throughout the body. Note also that ultraviolet light is not the only potential source of energy that can activate ACE pigments collected from a patient and placed in very close proximity to the patient's skin, or even within one or other of the patient's cavities or orifices.

Various devices and kits are envisioned that will facilitate the placement of the urine/dye mixture onto discrete skin lesions or other areas of the body. A slightly adhesive quality would be useful for covering lesions that are not in a horizontally supported position. A central window in the absorbent material could help in viewing an underlying lesion for fluorescence. A squeezable open urine collection bottle that could be subsequently fitted with a spray head or other dispensing nozzle could be designed and potentially pre-filled with a small quantity of preservative and odor-reducing agents.

The sun can also be used as an alternative source of ultraviolet light rather than relying on a light bulb and source of electricity. Even viewing the urine for dye inducible or direct fluorescence can be done in daylight using a glass or plastic filter that allows selective passage of UV light into a dark box in which the urine±dye, either alone or attached to a suitable carrier, such as a Q-tip, can be added.

The principles, preferred embodiments and modes of operation of the present invention have been described in the foregoing specification. The invention which is intended to be protected herein, however, is not to be construed as limited to the particular forms disclosed, since they are to be regarded as illustrative rather than restrictive. Additional advantages and modifications will readily occur to those skilled in the art. Variations and changes may be made without departing from the spirit of the invention encompassed by the appended claims.

What I claim as my invention is:

1. A method of activating the alternative cellular energy (ACE) pathway in a human or animal subject comprising the collection of urine from the patient; adding a quantity of dye, such as neutral red or acridine orange, to the ACE pigments contained within the urine so as to render them fluorescent or otherwise activated when illuminated with ultraviolet (UV) light; and having the mixture of activated ACE pigments and dye positioned onto a surface area of the body with continuing UV illumination in a manner that will activate the ACE pathway in the body and achieve a therapeutic benefit within the subject.

2. The method of claim 1 in which the illness being treated is autism.

3. The method of claim 1 in which the patient being treated is identified as having an insufficiency of cellular energy as determined by a more positive ultraviolet light inducible fluorescence reaction of a bodily fluid, such as urine and/or spittle (saliva) when the fluid is mixed with a solution of neutral red dye, than when the fluid is similarly examined in the absence of neutral red dye.

4. The method of claim 1 in which the condition being treated is a mental illness.

5. The method of claim 1 in which the illness is a neurological disease.

6. The method of claim 1 in which the condition is a respiratory, cardiovascular or cerebrovascular disease associated with an insufficiency of cellular energy.

7. The method of claim 1 in which the condition is a behavioral and/or learning disorder affecting a child or an adult.

8. The method of claim 1 in which the illness is the chronic fatigue syndrome and/or fibromyalgia.

9. The method of claim 1 in which the condition is referred to as delusional parasitosis or Morgellon's disease

10. The method of claim 1 in which the illness being treated is epilepsy

11. The method of claim 1 in which the condition being treated is either a localized or systemic infectious disease.

12. The method of claim 1 in which the dye used is neutral red at a concentration of between 0.01 to 5.0 milligrams per ml.

13. The method of claim 1 in which the dye used is acridine orange at a concentration of between 0.01 to 5.0 milligrams per ml.

14. The method of claim 1 in which the source of ultraviolet light is a lamp of between 5 and 200 watts that emits at a maximum wavelength between 350 and 380 nanometers.

15. The method of claim 1 in which sunlight is used as the source of ultraviolet illumination.

16. The method of claim 1 in which the activation of the body's ACE pathway is determined by the development during and/or following therapy of ultraviolet light inducible direct fluorescence within the patient's skin or in other areas of the body, including the mouth; and/or in bodily fluids, including urine, spittle and blood.

17. The method of claim 1 that comprises a kit on which to place the urine-dye mixture for placement onto the body.

18. The method of claim 1 that comprises a container for collecting urine and which may contain a preservative and/or an odor-reducing agent.
